(19) United States
(12) Patent Application Publication
(10) Pub. No.: US 2026/0268165 A1
(43) Pub. Date: Sep. 10, 2026

(54) Systems and Methods for Brain-Machine Interface Shared Autonomy

(71) Applicant: The Regents of the University of California, Oakland, CA (US)

(72) Inventors: Jonathan C. Kao, Los Angeles, CA (US); Brandon McMahan, Los Angeles, CA (US); Sangjoon Lee, Los Angeles, CA (US); Johannes Lee, Los Angeles, CA (US)

(73) Assignee: The Regents of the University of California, Oakland, CA (US)

(21) Appl. No.: 19/164,151

(22) PCT Filed: Mar. 14, 2024
(86) PCT No.: PCT/US2024/019967

§ 371 Date: Sep. 10, 2025

Related U.S. Application Data
(60) Provisional application No. 63/490,220, filed on Mar. 14, 2023.

(51) Int. Cl. G06N 3/10

(52) U.S. Cl. CPC G06N 3/10

(57) ABSTRACT

Systems and methods for brain-machine interfacing in accordance with embodiments of the invention are illustrated. One embodiment includes a brain-machine interface (BMI) with shared autonomy, including a neural signal recorder, a controller, including a processor, and a memory, where the memory contains a BMI application that configures the processor to obtain neural signals of a user via the neural signal recorder, decode the neural signals into a command for a connected device using a neural decoder model, obtain environmental state information describing an operating environment of the connected device, predict an intended goal of the user using a copilot model provided with the environmental state information and the command for the connected device, perform the intended goal using the connected device using a plurality of commands provided by the copilot model.

### CROSS-REFERENCE TO RELATED APPLICATIONS

The current application claims the benefit of and priority under 35 U.S.C. § 119 (e) to U.S. Provisional Patent Application No. 63/490,220 entitled “Systems and Methods for AI Assisted Brain-Machine Interfacing” filed Mar. 14, 2023. The disclosure of U.S. Provisional Patent Application No. 63/490,220 is hereby incorporated by reference in its entirety for all purposes.

### STATEMENT OF FEDERALLY SPONSORED RESEARCH

This invention was made with government support under NS122037 awarded by the National Institutes of Health. The government has certain rights in the invention.

### FIELD OF THE INVENTION

The present invention generally relates to brain-machine interfaces.

### BACKGROUND

Brain-Machine Interfaces (BMIs, sometimes referred to as Brain-Computer Interfaces, BCIs) are systems which translate neural signals recorded from a user's brain into commands for various connected prosthetic devices. A classic example of a BMI task is cursor control in a virtual keyboard. There are many different modalities for recording central and peripheral neural signals including (but not limited to) electroencephalography (EEG), electrocorticography (ECoG), magnetoencephalography (MEG), electromyography (EMG), and recording using microelectrode arrays. Different recording modalities produce different representations of neural activity.

### SUMMARY OF THE INVENTION

Systems and methods for brain-machine interfacing in accordance with embodiments of the invention are illustrated. One embodiment includes a brain-machine interface (BMI) with shared autonomy, including a neural signal recorder, a controller, including a processor, and a memory, where the memory contains a BMI application that configures the processor to obtain neural signals of a user via the neural signal recorder, decode the neural signals into a command for a connected device using a neural decoder model, obtain environmental state information describing an operating environment of the connected device, predict an intended goal of the user using a copilot model provided with the environmental state information and the command for the connected device, perform the intended goal using the connected device using a plurality of commands provided by the copilot model.

In a further embodiment, the neural signal recorder is an electroencephalography device.

In still another embodiment, the intended goal is movement of a cursor in a digital environment.

In a still further embodiment, the environmental state information includes a position of the cursor in the digital environment and locations of interactable objects in the digital environment.

In yet another embodiment, the intended goal is interaction with a given object with the connected device, wherein the connected device is a prosthetic arm.

In a yet further embodiment, the method further includes steps for a machine vision system, where the machine vision system is configured to generate the environmental state information, and where the environmental state information includes locations and positions of identified objects within reach of the prosthetic arm.

In another additional embodiment, the copilot model is a machine learning model.

In a further additional embodiment, the copilot model is trained using a synthetic softmax.

In another embodiment again, the copilot model includes an intervention function, where the intervention function configured to blend the command for the connected device generated by the neural decoder model, and the plurality of commands output by the copilot model.

In a further embodiment again, the connected device is a computer, and the intended command controls an avatar in a digital environment.

Another embodiment includes a method for brain-machine interfacing with shared autonomy, including recording neural signals of a user using a neural signal recorder, decoding the neural signals into a command for a connected device using a neural decoder model, obtaining environmental state information describing an operating environment of the connected device, predicting an intended goal of the user using a copilot model provided with the environmental state information and the command for the connected device, performing the intended goal using the connected device using a plurality of commands provided by the copilot model.

In still yet another embodiment, the neural signal recorder is an electroencephalography device.

In a still yet further embodiment, the intended goal is movement of a cursor in a digital environment.

In still another additional embodiment, the environmental state information includes a position of the cursor in the digital environment and locations of interactable objects in the digital environment.

In a still further additional embodiment, the intended goal is interaction with a given object with the connected device, wherein the connected device is a prosthetic arm.

In still another embodiment again, the method further includes steps for generating the environmental state information using a machine vision system, where the environmental state information includes locations and positions of identified objects within reach of the prosthetic arm.

In a still further embodiment again, the copilot model is a machine learning model.

In yet another additional embodiment, the copilot model is trained using a synthetic softmax.

In a yet further additional embodiment, the copilot model includes an intervention function, where the intervention function configured to blend the command for the connected device generated by the neural decoder model, and the plurality of commands output by the copilot model.

In yet another embodiment again, the connected device is a computer, and the intended command controls an avatar in a digital environment. Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a brain-machine interface system in accordance with an embodiment of the invention.

FIG. 2 illustrates another brain-machine interface system in accordance with an embodiment of the invention.

FIG. 3 is a block diagram for a controller in accordance with an embodiment of the invention.

FIG. 4 is a flow chart for a brain-machine interfacing process using a copilot in accordance with an embodiment of the invention.

FIG. 5 is a flow chart for a brain-machine interfacing process using a copilot for controlling a virtual cursor in accordance with an embodiment of the invention.

FIG. 6 is a flow chart for a brain-machine interfacing process using a copilot for controlling a prosthetic arm in accordance with an embodiment of the invention.

FIG. 7 is a diagram showing the operation of an analytical copilot model for robot arm control in accordance with an embodiment of the invention.

FIG. 8 is a flow diagram showing operation steps of brain-machine interface in accordance with an embodiment of the invention.

FIG. 9A is a flow diagram showing operation steps of a brain-machine interface in accordance with an embodiment of the invention.

FIG. 9B is a flow diagram showing training steps of a brain-machine interface in accordance with an embodiment of the invention.

### DETAILED DESCRIPTION

Over 5 million people in the USA—nearly 1 person in 50—live with a form of paralysis due to causes including stroke, spinal cord injury, multiple sclerosis, and ALS. People who have lost the ability to move also lose a profound sense of control, freedom, and independence in their lives. But paralysis does not take away one's intent or desire to move; the brain still encodes these thoughts through neural signals. Brain-machine interfaces (BMIs) aim to restore the ability to communicate with the world by translating these neural signals into actions. BMIs decode neural signals into the movements of a computer cursor on a screen or a robotic arm, allowing the user to interact with the world autonomously. While BMIs have existed for over two decades, they have remained in pilot clinical trials dating back to 2004. Intracortical BMI clinical trials remain in a research phase and have not achieved widespread use. A key reason for this is that BMI performance has not achieved levels of performance that overcome their costs and risks. This is true for both invasive BMIs requiring neurosurgery, as well as for non-invasive BMIs, which can be used without surgical procedures but currently achieve lower performance.

Systems and methods described herein use a trained artificial intelligence called a “copilot” to synergistically aid a user in performing tasks. Using a copilot as described herein leads to a categorical increase in BMI performance. In many embodiments, copilots are trained to learn structures and patterns in the tasks users do, enabling it to control the machine when it is confident in the user's intention. For example, consider taking a drink from a cup on a table. Using today's state-of-the-art BMIs, the user would need to neurally control the minutiae of a moment-by-moment robotic arm trajectory towards the cup, grasp the cup with appropriate pressure, and bring the cup to their mouth. This task is difficult, and BMIs today perform these actions well below naturalistic speeds. When using a copilot, when it becomes clear that the user is attempting to pick up a cup, the robotic arm can be automatically directed to pick up and grasp the cup, thereby reducing the “neural effort” of the task.

In order to make a confident prediction of the user's intention, the copilot can be given access to data describing the state of the user's operating environment. For example, in a simple cursor control environment, the operating environment can be presented as key locations, the current location of the cursor, and/or previous inputs, all of which are immediately known to the computer operating environment. More complex computing environments, i.e. controlling an avatar in a digital environment, may provide additional environmental information. With completely digital environments, it can be relatively easy to obtain information about the environment. Physical environments may be sensed by more complex sensing tools. For example, in the context of a robotic arm, a machine vision system can be used to identify and classify objects in the environment, as well as determine their spatial coordinates relative to the robotic arm. As can be readily appreciated, there are any number of environments in which a user may want to operate, and additional sensors can be added that are specialized for a given environment as appropriate to the requirements of specific applications of embodiments of the invention.

In many embodiments, the copilot has access to live video data and uses computer vision to identify items on the table, including the cup. The copilot can then compute possible actions with identified items. In this case, humans typically drink from a cup after picking it up. In various embodiments, the copilot has access to neural information (e.g., the user may command movement in the general direction of the cup). The copilot, inferring the user wants a drink, can then precisely complete the action. Instead of the user neurally commanding a detailed robotic arm trajectory, a slow and high-effort process, the copilot would help the user complete the action quickly, seamlessly, and efficiently by taking over control. This is an example of a concept referred to as “shared autonomy”, where a user decides and sets a goal, and an agent (in this case the copilot) is delegated control to achieve that goal. In some embodiments, even if a copilot cannot determine what the user's goal is, it can take control in order to prevent a universally bad state, i.e. preventing a vehicle from colliding with a wall. Different levels of shared autonomy are possible, where different levels of detail are assigned to the user and to the agent. As can be readily appreciated, grasping a cup is merely an example, and copilots can be trained to perform any number of different tasks for users. By way of further example, when using a computer, a copilot can infer what icons you wish to select with a computer mouse based on past movements and neural information. When interacting generally with objects, a copilot can be trained as to how the user typically interacts with them (e.g., if a computer vision module observes a handbag and wallet, and the user moves towards the wallet, it is likely the user intends to put the wallet in the handbag).

In many embodiments, scalp potentials recorded by electroencephalography (EEG) is used as the neural signal to control the BMI and/or copilot. However, any number of different neural recording modalities can be used including (but not limited to) intracortical spiking recorded using implanted microelectrode arrays, electrocorticography (ECOG), and/or any other modality as appropriate to the requirements of specific applications of embodiments of the invention. Various BMI systems are discussed below, followed by methods of their use.

### Shared Autonomy BMI Systems

Shared autonomy BMI systems can have many different architectures depending on the tasks to be performed. If an operating environment is all digital, physical sensors may be less important than in a physical environment. Further, neural signal recorders may be different depending on the user. For example, some users may be implanted with microelectrode arrays for precise neural recordings, whereas others may use a non-invasive modality. Depending on the specific task and/or numerosity of the tasks, the resolution required for a given system may be considerably different. Even for highly complex tasks, if there are only two specific tasks, it may be sufficient for a well-trained copilot with significant autonomy to merely be instructed between the two. As the number of tasks rises, and/or the complexity of the user input rises, it may be more beneficial to have higher resolution recording modalities for faster, more accurate performance.

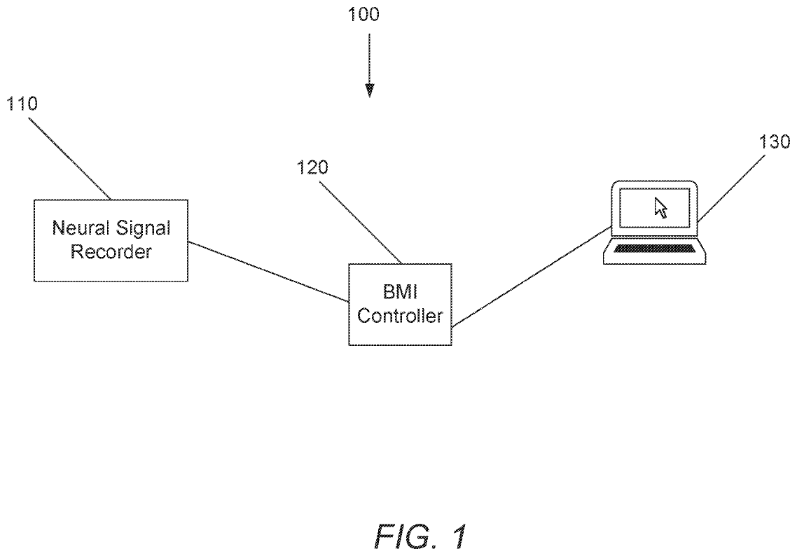

Turning now to FIG. 1, an example shared autonomy BMI system in accordance with an embodiment of the invention is illustrated. System 100 includes a neural signal recorder 110. As previously noted, neural signal recorders can be any number of different devices depending on the recording modality desired. Neural signals are transmitted to the BMI controller 120, which is configured to decode the neural signals and to operate the copilot model. The BMI controller is connected to a computer running a digital operating environment with a controllable cursor 130. In numerous embodiments, the digital operating environment has different controllable objects such as (but not limited to) a digital avatar.

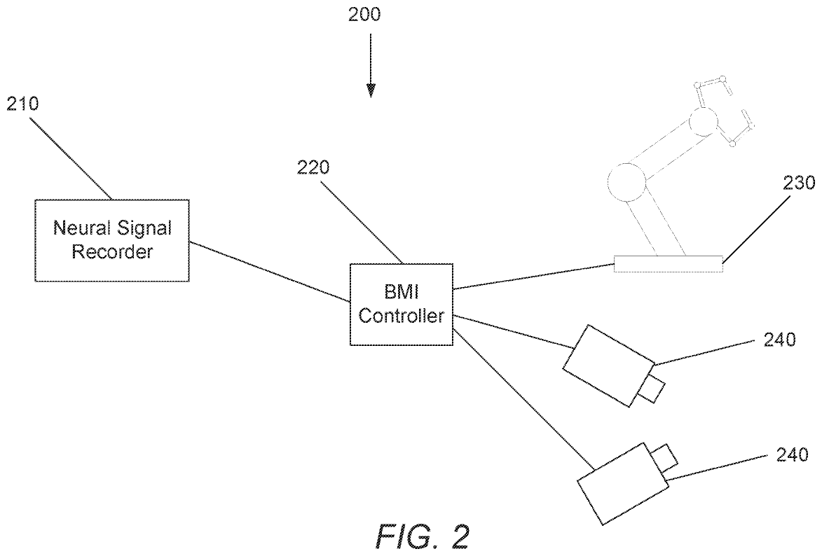

FIG. 2 provides a second example shared autonomy BMI system in accordance with an embodiment of the invention. System 200 includes a neural signal recorder 210, and a BMI controller 220. The BMI controller is connected to a prosthetic device, in this case a robotic arm 230. The BMI controller is also connected to a machine vision system 240. In many embodiments, machine vision systems incorporate their own computing hardware for performing object detection and other machine vision processes. However, in many embodiments, the BMI controller is additionally configured to take in video streams and carry out the machine vision processes as illustrated in FIG. 2.

As can readily be appreciated, any number of different architectures are possible depending on a given task. For example, mobile robot or drone control may have many additional environmental sensors on-board or in the environment in order to properly model the environmental state. Similarly, different neural recording devices can be used depending on the recording modality.

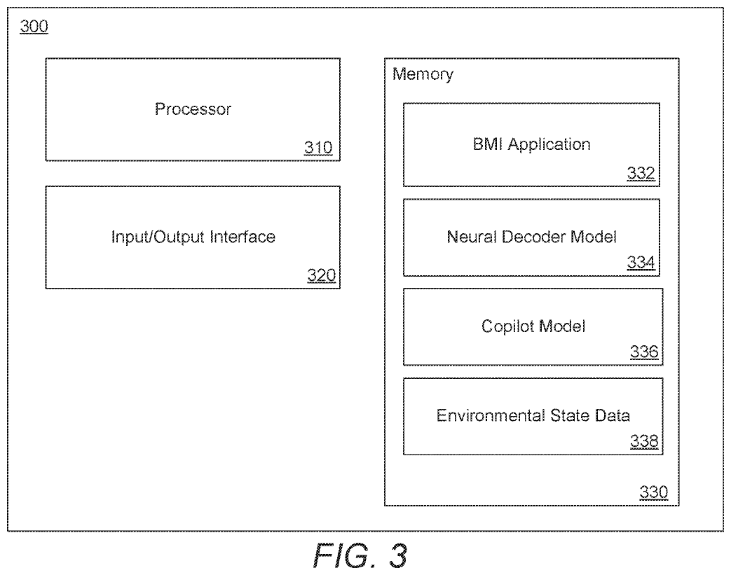

Turning now to FIG. 3, a BMI controller in accordance with an embodiment of the invention is illustrated. BMI controller 300 includes a processor 310. In many embodiments, the processor is one or more logic processing circuitries including (but not limited to) a central processing unit (CPU), a graphics processing unit (GPU), an application-specific integrated circuit (ASIC), a field-programmable gate array (FPGA), and/or any other logic processing circuitry capable of performing processes described herein as appropriate to the requirements of specific applications of embodiments of the invention. Controller 300 further includes an input/output (I/O) interface 320. In many embodiments, I/O interfaces are used to communicate with other devices such as (but not limited to) neural signal recorders, prosthetic devices, and/or any other connected prosthetic device as appropriate to the requirements of specific applications of embodiments of the invention.

Controller 300 additionally includes a memory 330. Memories can be made of volatile memory, non-volatile memory, and/or any combination thereof. Memory 330 contains a BMI application which can configure the processor to carry out various shared autonomy BMI processes such as (but not limited to) generating control signals for connected prosthetic devices based on recorded neural signals. Memory 330 at various times may include a neural decoder model 334 which translates neural signals to commands for a given connected prosthetic device, and a copilot model 336 that is trained to generate commands to complete predicted tasks based on the output of the neural decoder model and the environmental state, stored as environmental state data 338. As can be readily appreciated, any number of computing platforms can be used as a BMI controller as long as sufficient computing resources are available. Methods for brain-machine interfacing with shared autonomy are further described below.

### Shared Autonomy Brain-Machine Interfacing

A goal of shared autonomy BMIs is to reduce the neural work required for a given task. To do so systems and methods described herein utilize a copilot model that is used to offload the mechanics of a given task once that given task is identified. Doing so reduces the neural work required by a user to perform a given task. In many embodiments, a neural decoder model is trained using data from the specific end-user. Similarly, the copilot can be trained to perform desired tasks based on the specific end-user. However, in many embodiments, the copilot can be trained using crafted “expert” protocols for given tasks to enhance performance.

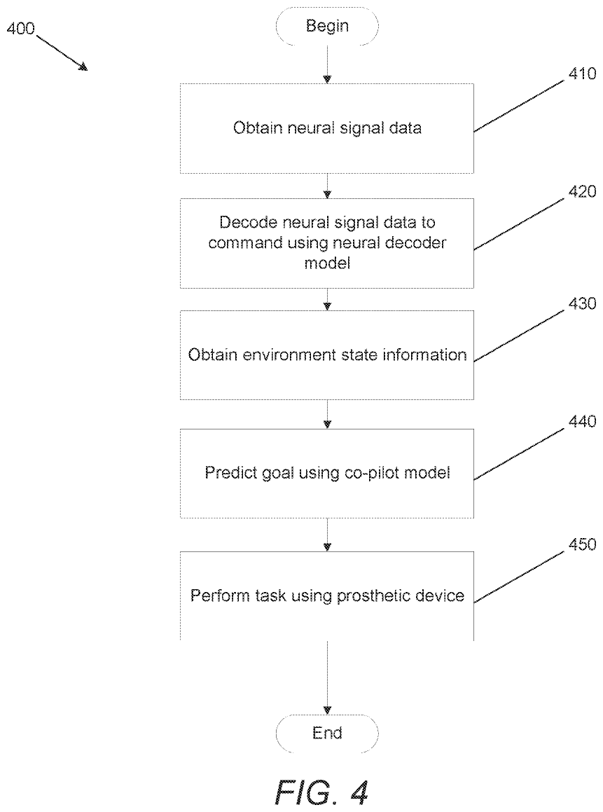

Turning now to FIG. 4, a process for brain-machine interfacing with shared autonomy in accordance with the invention is illustrated. Process 400 includes obtaining (410) neural signal data from a user. As noted above, any number of different modalities can be used to obtain neural signal data. The neural signal data is decoded (420) into a command using a neural decoder model. In numerous embodiments, the neural decoder model is a machine learning model which is trained to decode neural signals recorded using the selected modality to commands for a given connected prosthetic device. Information about the state of the operating environment is obtained (430). In numerous embodiments, the environmental state is recorded using sensors, i.e. a machine vision system, or any other appropriate sensor. In various embodiments, the environmental state includes information about the position of the controlled device in the environment, as well as the position and/or orientations of objects in the environment.

The user's intended goal is predicted (440) using the copilot model by providing the copilot model with the environmental state information as well as the output of the neural signal decoder. In numerous embodiments, the output of the copilot model includes a set of commands for the connected prosthetic device which when performed (450) carry out the intended task. In many embodiments, the set of commands is more complex than the output of the neural decoder model. In various embodiments, the set of commands may be the input command if the copilot is not certain which goal is intended, and the input command is not immediately deleterious. As can be readily appreciated, the prosthetic device can be any arbitrary device, either digital or physical, which is being controlled by the BMI, and is not restricted to conventional limb prosthetics. For example, a virtual avatar, a cursor, a car, a drone, a robot, or any other device can be controlled by a BMI and therefore be considered a “prosthetic device”.

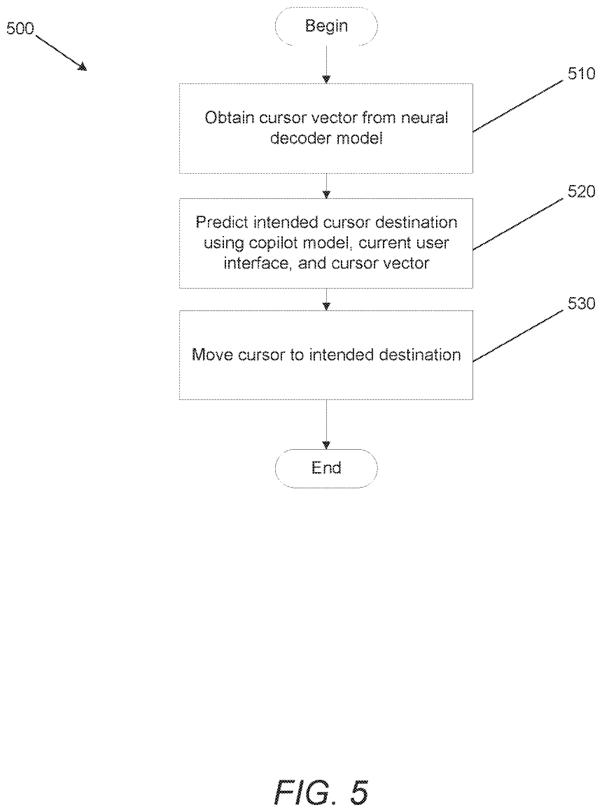

To contextualize the above, FIG. 5 is a process for controlling a virtual cursor in accordance with an embodiment of the invention. Process 500 includes obtaining a cursor vector (510) from a neural decoder model. The copilot then predicts (520) the intended cursor destination using the current cursor position, position of interactable objects in the environment, and the cursor vector. The cursor is then moved (530) to the predicted intended position, rather than simply moving it in accordance with the decoded cursor vector.

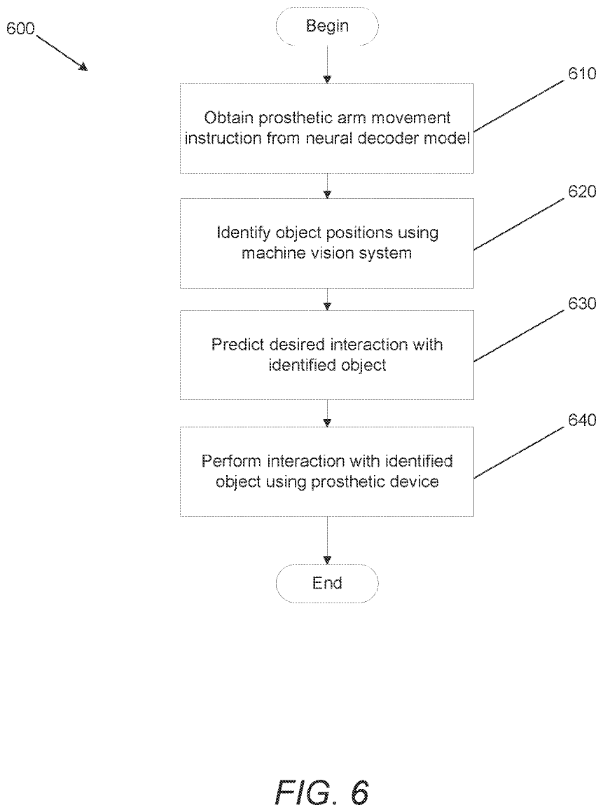

For additional context in a more complex, physical environment, FIG. 6 is a process for controlling a prosthetic robot arm in accordance with an embodiment of the invention. Process 600 includes obtaining (610) a movement instruction for the prosthetic arm using the neural decoder model. In many embodiments, this is a vector that indicates an immediate movement for the arm with respect to one or more articulations. However, the instruction can be represented in any number of ways as appropriate to the requirements of specific applications of embodiments of the invention. A machine vision system is used to identify (620) objects and their positions in the operating environment of the prosthetic arm. The copilot is provided with information from the machine vision system, as well as the decoded movement instructions. In various embodiments, the machine vision system provides at least identified objects and their positions. In some embodiments, the machine vision system provides access to intermediate feature maps as a latent representation of the goal space. The copilot predicts (630) an intended interaction with a particular object based on the decoded instructions and proceeds to perform (640) the predicted action without the need for further input.

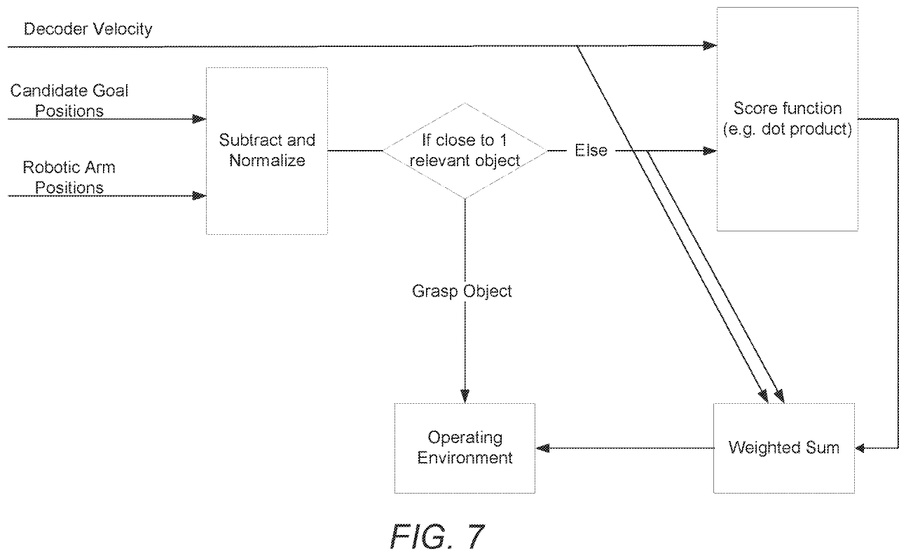

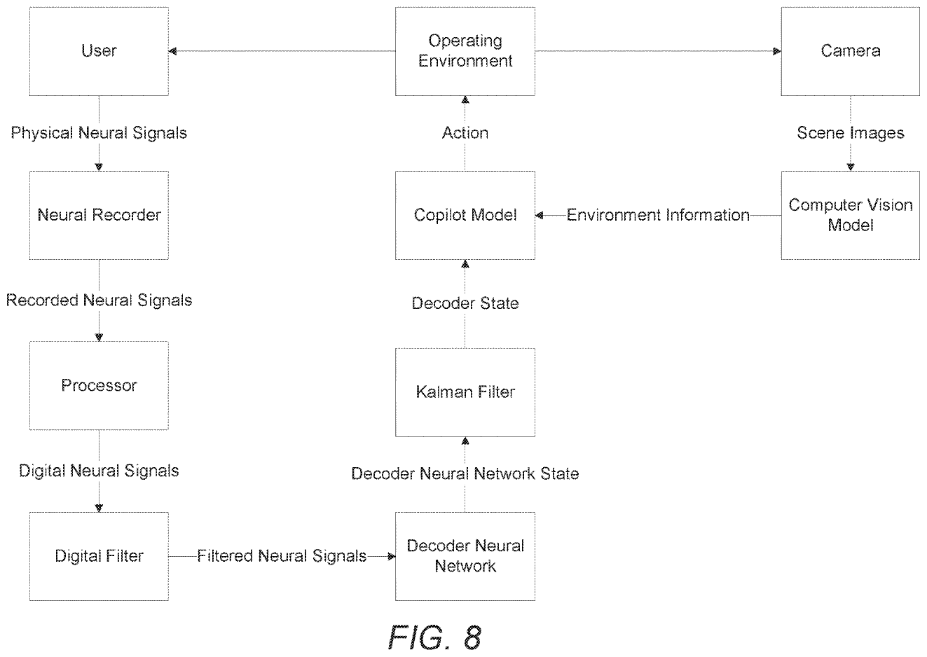

Depending on the tasks to be performed, the specific implementation of the neural decoding model and the copilot can be modified. For example, the copilot model need not be a machine learning model. FIG. 7 illustrates an analytical copilot model in accordance with an embodiment of the invention. In numerous embodiments, the neural decoding model is trained using data from tasks where the user performs prompted discrete actions at each point in time. In numerous embodiments, a Kalman filter can be used to filter input neural signals, and to ensure compatibility, the neural decoder model can be trained with regression (mean squared error loss) to predict one-hot representations of each discrete action. Training data can be generated from users performing tasks in either open-loop operation, closed-loop operation, or both, where weights are individualized for each user. The Kalman filter can be initially trained using the same data used to train the neural signal decoder, but can be allowed to update its parameters during user tasks using Closed Loop Decoder Adaptation. FIG. 8 is a flow diagram including use of a Kalman filter for robotic arm control in accordance with an embodiment of the invention. However, as can readily be appreciated, there are many different neural decoder models which are suited for specific tasks, any of which could be used as appropriate to the requirements of specific applications of embodiments of the invention.

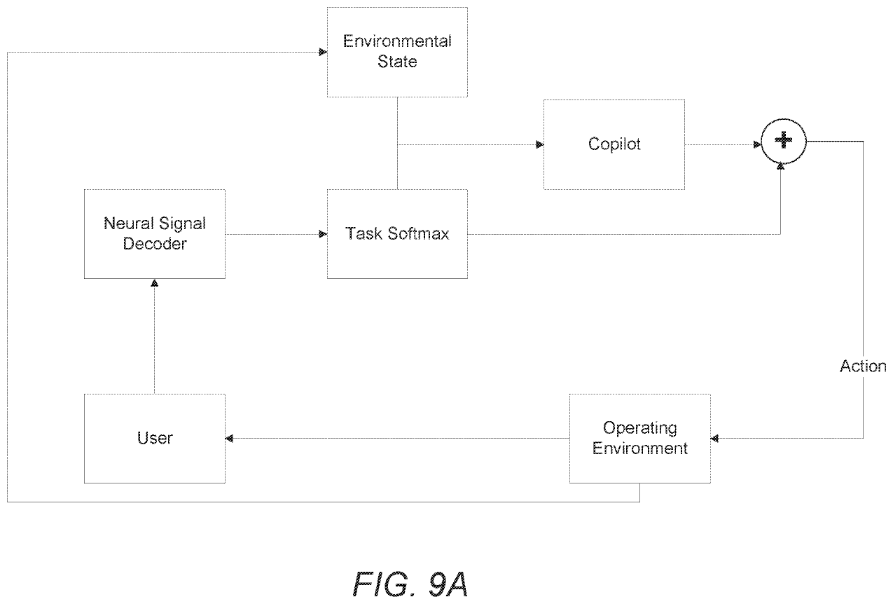

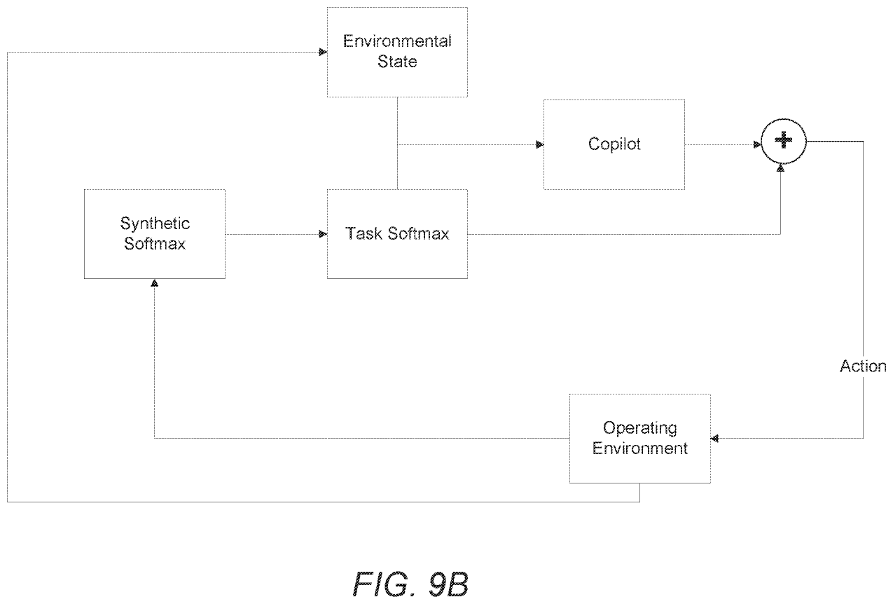

In many embodiments, in order to reduce training time where a human must be in the loop, a synthetic softmax can be used as a stand-in for a human user during training. Turning now to FIGS. 9A and 9B, a two diagrams illustrating the operation phase of an in-use BMI and a training phase of a BMI in accordance with an embodiment of the invention are illustrated. As shown, the user and neural signal decoder that are part of the operation phase can be replaced with a synthetic softmax during training.

In the context of cursor control, the copilot can accept as input a cursor's kinematics and decoder output (the softmax probabilities of each class) to predict a continuous cursor movement velocity. In various embodiments, the softmax probabilities are constructed as a synthetic softmax by creating a one-hot encoding vector with added noise. For example, in the case of 2D cursor control with four cardinal movements (up, down, left right), to generate a synthetic softmax, let (x,y) be the desired direction of the cursor based on a target. The synthetic decoder output v: = [−x,x,−y,y], where vᵢ=1 if vᵢ==max(v), else vi=0. Let v+U(0,1)*m+k, where U(0,1) is the uniform distribution with scaling m and bias k. In a number of embodiments, p (output| environment) can alternatively be modeled using long-term short-term networks (LSTM) and a generative adversarial network (GAN). The LSTM can be used to model change in softmax over time, and the GAN is used to generate fake softmax data.

Again in the context of cursor control, in various embodiments, a normalized vector pointing from the simulated cursor to the simulated target is calculated (an “intended direction vector”) periodically. Then noise can be added to the intended direction vector (e.g., perturb angle and norm). The noisy intended direction vector can then be sampled from a region around the perturbed intended direction vector (e.g. within a radius r) during a given period. From the noisy intended direction vector, a 4-class softmax vector is generated and noise can be added again softmax vector. As can readily be appreciated, there are many different ways to generate synthetic softmax vectors without departing from the scope or spirit of the invention, including those outside the scope of cursor control.

In various embodiments, the copilot can also accept target positions; task information (such as whether the cursor is in the acquisition window of a target); the output of other predictors; or the actual neural data. For example, in a number of embodiments, the copilot is a neural network that is trained via deep reinforcement learning (RL) such as actor-critic networks to enable the copilot to output continuous actions. The neural network accepts the copilot inputs and generates the velocity and/or acceleration of the computer cursor. In various embodiments, the copilot is trained to maximize rewards for performing the task. Positive rewards can be given for approaching and acquiring the correct target. In some embodiments, it is beneficial to give positive reinforcement for slow velocities within the target acceptance window. Negative rewards can be given for the passage of time and incorrect target selection.

In a variety of embodiments, curriculum learning is used to train the copilot. For example, when training to perform cursor control, the target size is reduced by a factor whenever the success rate reaches over 90%. By way of further example, in robotic arm control, the copilot is trained via deep reinforcement learning to move towards and grasp objects. It can use an image processing neural network to continuously process images of the scene and identify the positions of objects. It then processes the output of the neural decoder to move towards particular objects and grasp them. In another instantiation, the softmax of the neural network decoder is used to specify a latent variable that is then translated into robotic arm movements.

With respect to the copilot architecture, many different implementations are contemplated. In various embodiments, the specific implementations may change across tasks. For example, trajectory based tasks may be performed with multilayer perceptrons. Convolutional neural networks can also be used. In various embodiments, temporal copilots that take into account long-term history of environment and/or prosthetic device state may use long-term short-term layers as their last layer. Indeed, any number of different machine learning architectures, including other forms of recurrent neural networks, or neural networks generally, can be used as the foundation of a copilot depending on the specific needs of the set of tasks to be performed in a given environment or set of environments. In many embodiments, generative adversarial networks (GANs) can be used to create Pθ (output| context) that matches real pairs of (decoder output, context) to enable the generation of additional training data.

Deciding point at which the copilot takes over (and the manner in which it does so) is a complex question. Previous works have utilized a fixed hyper-parameter that must be set by the implementer or system operator, while others have required that their copilot directly compute an offset to the human action instead of generating its own assistive action. In many embodiments, that decision point is formulated under a framework for shared autonomy referred to herein as “interventional assist”, or IA. IA seeks to dynamically blend the action of the copilot with the action of the human operator.

IA contains three main components: a human that generates a control signal, an assistive copilot that generates a suggested action, and an intervention function that determines how to blend the human and copilot for effective control. In many embodiments, the control signal is the output of the neural decoder model which is fed neural signals recorded from the human user. In numerous embodiments, the copilot model is trained using “expert” demonstrations of given tasks. The way these demonstrations are collected does not matter for training a diffusion process, i.e. they could be policy rollouts from a trained expert, provided by an actual human, or be algorithmically generated (i.e. with a generative model).

A set of expert demonstrations that consists of state action pairs is formalized as:

$$D = ( {s}_{0} , {a}_{0 )} ) , ( {s}_{1} , {a}_{1 )} ) , ... ( {s}_{N} , {a}_{N} )$$

In many embodiments, goal specific information is removed from the state. For example, in the context of a cursor control task, any information about the location of the target from the state, s, is removed, and the goal agnostic state is denoted s.

A diffusion process can then be trained to recover [ŝᵢ|aᵢ] after adding successive amounts of noise to a. In various embodiments, the added noise is Gaussian noise. In a number of embodiments, the noise is sampled from a human using a neural decoder to perform the given task. However, any number of different noise addition approaches can be taken as appropriate to the requirements of specific applications of embodiments of the invention. This diffusion process, in turn, learns to push noisy actions closer to those seen in expert demonstrations. During human-in-the-loop control using the trained model, the copilot treats a human action as if it is noisy and pushes it closer to an expert demonstration. Because the goal is internal to the human and not known to the copilot, the diffusion process can be trained on partial state observations, s. This forces the diffusion process to infer goal intent from the action a of the human without being able to explicitly read goal information.

In order to dynamically blend human and copilot actions, the expected future return associated with following the copilot action vs the human action is analytically determined in a goal agnostic fashion. The intervention function is formalized as:

$$I ( {a}_{c} , {a}_{h} \vert \hat{s} ) = {\int }_{\hat{g} ∈ G *} Q ( \hat{s} , {a}_{c} , \hat{g} ) d \hat{g} - Q ( \hat{s} , {a}_{h} , \hat{g} ) d \hat{g}$$

The intervention function represents the difference in expected future return of following the copilot vs the human from the current goal agnostic state s. Because the state-action function, Q, depends on the goal, which is something internal to the human and cannot easily be known in the real world, the goal is marginalized in favor of integrating over all possible goals. In various embodiments, Q is obtained from an expert trained to perform the given task. In many embodiments, Q is obtained from demonstrations collected while users perform the task. In various embodiments, ensembles of Q networks that come from multiple sources are used. This results in a sense of how much better, in expectation over all goals, the copilot's suggested action, a_{c}, is compared to the human's intended action, aₕ. Once I (a_{c}, aₕ) is computed, the two actions can be blended together. In many embodiments, the blending involves simply choosing one action or another when the value of one action is higher. In some embodiments, a convex combination weighted proportionally to the intervention is calculated. In various embodiments, a margin is set and if the margin is exceeded, the high valued action is selected, otherwise take a convex combination of the actions. Indeed, any number of blending strategies can be taken depending on the user and the tasks to be performed.

Further, the intervention function can be extended such that it considers the entire past trajectory through the operating environment. By considering an entire history of state transitions, the intervention function may better assess which current action is likely to have higher expected return in the future while maintaining consistency with past actions. This modified intervention function is formalized as:

$$I ( {a}_{c} , {a}_{h} ) = {\int }_{\hat{g} ∈ G *} V ( ( {s}_{0} , {a}_{0} ) , ... , ( {s}_{t} , {a}_{c} ) \vert \hat{g} ) d \hat{g} - V ( ( {s}_{0} , {a}_{0} ) , ... , ( {s}_{t} , {a}_{h} ) \vert \hat{g} ) d \hat{g}$$

An additional modification can be made in order to restrain the goal space in situations where it can reliably be determined what are the most likely goals in the current environment. For example, in the case of cursor control in a 2D environment, all possible goal positions can be determined by the location of every clickable object on the screen. In the case of robotic arm control, a machine vision system with real-time object detection can determine the location of all objects that could possibly be grasped by the arm given the current location.

As can readily be appreciated, the concept of a copilot which is incorporated into a BMI can greatly improve performance irrespective of task. The exact form of the copilot can be dependent upon the training regime and machine learning model selected. However, any number of different training regimes and machine learning models can be used without departing from the scope or spirit of the invention. In numerous embodiments, the copilot and neural decoder are trained to perform any number of different tasks that a user may want to perform. Similarly, any number of different sensors can be incorporated to provide data to assist with the copiloting process. While video data is discussed above, different optical sensors, temperature sensors, pressure sensors, hygrometers, microphones, and/or any other different type of sensor can be incorporated as appropriate to the requirements of specific applications of embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A brain-machine interface (BMI) with shared autonomy, comprising:
a neural signal recorder;
a controller, comprising:
a processor; and
a memory, where the memory contains a BMI application that configures the processor to:
obtain neural signals of a user via the neural signal recorder;
decode the neural signals into a command for a connected device using a neural decoder model;
obtain environmental state information describing an operating environment of the connected device;
predict an intended goal of the user using a copilot model provided with the environmental state information and the command for the connected device;
perform the intended goal using the connected device using a plurality of commands provided by the copilot model.

2. The BMI with shared autonomy of claim 1, wherein the neural signal recorder is an electroencephalography device.

3. The BMI with shared autonomy of claim 1, wherein the intended goal is movement of a cursor in a digital environment.

4. The BMI with shared autonomy of claim 3, wherein the environmental state information comprises a position of the cursor in the digital environment and locations of interactable objects in the digital environment.

5. The BMI with shared autonomy of claim 1, wherein the intended goal is interaction with a given object with the connected device, wherein the connected device is a prosthetic arm.

6. The BMI with shared autonomy of claim 5, further comprising a machine vision system, where the machine vision system is configured to generate the environmental state information, and where the environmental state information comprises locations and positions of identified objects within reach of the prosthetic arm.

7. The BMI with shared autonomy of claim 1, wherein the copilot model is a machine learning model.

8. The BMI with shared autonomy of claim 7, wherein the copilot model is trained using a synthetic softmax.

9. The BMI with shared autonomy of claim 1, wherein the copilot model comprises an intervention function, where the intervention function configured to blend the command for the connected device generated by the neural decoder model, and the plurality of commands output by the copilot model.

10. The BMI with shared autonomy of claim 1, wherein the connected device is a computer, and the intended command controls an avatar in a digital environment.

11. A method for brain-machine interfacing with shared autonomy, comprising:
recording neural signals of a user using a neural signal recorder;
decoding the neural signals into a command for a connected device using a neural decoder model;
obtaining environmental state information describing an operating environment of the connected device;
predicting an intended goal of the user using a copilot model provided with the environmental state information and the command for the connected device;
performing the intended goal using the connected device using a plurality of commands provided by the copilot model.

12. The method for brain-machine interfacing with shared autonomy of claim 11, wherein the neural signal recorder is an electroencephalography device.

13. The method for brain-machine interfacing with shared autonomy of claim 11, wherein the intended goal is movement of a cursor in a digital environment.

14. The method for brain-machine interfacing with shared autonomy of claim 13, wherein the environmental state information comprises a position of the cursor in the digital environment and locations of interactable objects in the digital environment.

15. The method for brain-machine interfacing with shared autonomy of claim 11, wherein the intended goal is interaction with a given object with the connected device, wherein the connected device is a prosthetic arm.

16. The method for brain-machine interfacing with shared autonomy of claim 15, further comprising generating the environmental state information using a machine vision system, where the environmental state information comprises locations and positions of identified objects within reach of the prosthetic arm.

17. The method for brain-machine interfacing with shared autonomy of claim 11, wherein the copilot model is a machine learning model.

18. The method for brain-machine interfacing with shared autonomy of claim 17, wherein the copilot model is trained using a synthetic softmax.

19. The method for brain-machine interfacing with shared autonomy of claim 11, wherein the copilot model comprises an intervention function, where the intervention function configured to blend the command for the connected device generated by the neural decoder model, and the plurality of commands output by the copilot model.

20. The method for brain-machine interfacing with shared autonomy of claim 11, wherein the connected device is a computer, and the intended command controls an avatar in a digital environment.
